(19) United States
(12) Patent Application Publication
(10) Pub. No.: US 2026/0337458 A1
(43) Pub. Date: Oct. 29, 2026

(54) ARTICLE COMPRISING ADHESIVE AND SHELL

(71) Applicant: Solventum Intellectual Properties Company, Maplewood, MN (US)

(72) Inventors: Audrey A. SHERMAN, Saint Paul, MN (US); Kristin J. GODBEY, Saint Paul, MN (US); Dylan T. COSGROVE, Saint Paul, MN (US); Anne C. F. GOLD, Saint Paul, MN (US); Abhishek SRIVASTAVA, Saint Paul, MN (US); Thomas R. CORRIGAN, Saint Paul, MN (US); Jingyi GUO, Saint Paul, MN (US); Chaodi LI, Saint Paul, MN (US); Gene P. MCNAMARA, Saint Paul, MN (US)

(73) Assignee: Solventum Intellectual Properties Company, Maplewood, MN (US)

(21) Appl. No.: 19/116,192

(22) PCT Filed: Oct. 9, 2023
(86) PCT No.: PCT/IB2023/060119

§ 371 Date: Mar. 27, 2025

Related U.S. Application Data
(60) Provisional application No. 63/379,201, filed on Oct. 12, 2022.

(51) Int. Cl. C09J 7/20; C09J 5/00

(52) U.S. Cl. CPC C09J 7/203; C09J 5/00; C09J 2301/204; C09J 2301/302; C09J 2301/308

(57) ABSTRACT

An article is described that includes at least one adhesive layer including an adhesive. The article further includes at least one shell enclosing the at least one adhesive layer. The at least one shell incudes a main member including a first major surface, a second major surface opposite to the first major surface, and a perimeter disposed between the first major surface and the second major surface. The second major surface is coupled to the at least one adhesive layer. The at least one shell further includes a side member extending from and coupled to at least a portion of the perimeter of the main member. The side member extends from the perimeter beyond the second major surface of the main member and at least partially surrounds the at least one adhesive layer. The side member is configured to flex relative to the main member.

### TECHNICAL FIELD

The present disclosure generally relates to an article including an adhesive and a shell.

### BACKGROUND

Articles that include adhesive layers are generally found in a variety of applications. Such articles may be held against an external surface with the help of the adhesive layer. For example, medical diagnostic devices are typically held against the skin of a user through an adhesive layer. Such devices need to be attached to the external surface (e.g., the skin of the user) that is not rigid and generally not flat. Additionally, such devices tend to catch on external obstacles and get pulled off the skin of the user, e.g., through edge lifting. Once the device falls off the user, diagnostic data may not be collected, and a replacement device may not be always available. Further, the original device may not be reattached due to sterility issues or a loss of function. Thus, articles that may last longer on the external surface (e.g., the skin of the user) may be desirable.

It is known to provide articles (i.e., medical diagnostic devices) that protect the device from external obstacles. An example of such article is provided in U.S. Pat. No. 11,219,413 (hereinafter referred to as '413 reference). The '413 reference discloses an adhesive pad system that includes a reinforcing overlay. The reinforcing overlay at least partially covers the adhesive pad. The reinforcing overlay may be removable without disturbing a sensor so that the reinforcing overlay may be replaceable.

### SUMMARY

In one aspect, an article is described. The article includes at least one adhesive layer including an adhesive. The article further includes at least one shell enclosing the at least one adhesive layer. The at least one shell incudes a main member including a first major surface and a second major surface opposite to the first major surface. The second major surface is coupled to the at least one adhesive layer. The main member further includes a perimeter disposed between the first major surface and the second major surface. The at least one shell further includes a side member extending from and coupled to at least a portion of the perimeter of the main member. The side member extends from the perimeter beyond the second major surface of the main member and at least partially surrounds the at least one adhesive layer. The side member is configured to flex relative to the main member.

In another aspect, a method of using the article of the first aspect is described. The method includes bonding the at least one adhesive layer to an external surface and engaging the side member with the external surface.

The details of one or more examples of the disclosure are set forth in the accompanying drawings and the description below. Other features, objects, and advantages of the disclosure will be apparent from the description and drawings, and from the claims.

### BRIEF DESCRIPTION OF DRAWINGS

Exemplary embodiments disclosed herein may be more completely understood in consideration of the following detailed description in connection with the following figures. The figures are not necessarily drawn to scale. Like numbers used in the figures refer to like components. However, it will be understood that the use of a number to refer to a component in a given figure is not intended to limit the component in another figure labeled with the same number.

FIG. 1A is a schematic perspective view of an article, according to an embodiment of the present disclosure;

FIG. 1B is a schematic sectional side view of the article, according to an embodiment of the present disclosure;

FIG. 2 is a schematic sectional side view of the article, according to another embodiment of the present disclosure;

FIG. 3 is a schematic sectional side view of the article, according to yet another embodiment of the present disclosure;

FIG. 4A is a schematic perspective view of an article, according to another embodiment of the present disclosure;

FIG. 4B is a schematic bottom view of the article of FIG. 4A, according to an embodiment of the present disclosure;

FIG. 5 is a schematic sectional side view of the article of FIG. 4A, according to an embodiment of the present disclosure;

FIG. 6 is a schematic sectional side view of the article of FIG. 4A, according to another embodiment of the present disclosure;

FIG. 7A is a schematic sectional side view of an article, according to another embodiment of the present disclosure;

FIG. 7B is an enlarged schematic view of a section S1 of the article shown in FIG. 7A, according to an embodiment of the present disclosure;

FIG. 8A is a schematic sectional side view of an article, according to another embodiment of the present disclosure;

FIG. 8B is an enlarged schematic view of a section S2 of the article shown in FIG. 8A, according to an embodiment of the present disclosure;

FIG. 9 is a schematic perspective view of an article, according to another embodiment of the present disclosure;

FIG. 10 is a schematic perspective view of an article, according to another embodiment of the present disclosure;

FIG. 11 is a schematic sectional view of the article taken along a section line A-A′ shown in FIG. 10, according to an embodiment of the present disclosure;

FIG. 12 is a schematic perspective view of an article, according to another embodiment of the present disclosure;

FIG. 13 is a schematic sectional view of the article taken along a section line B-B′ shown in FIG. 12, according to an embodiment of the present disclosure;

FIG. 14 is a schematic sectional view of the article taken along the section line B-B′ shown in FIG. 12, according to another embodiment of the present disclosure;

FIG. 15 is a schematic sectional view of the article taken along the section line B-B′ shown in FIG. 12, according to yet another embodiment of the present disclosure;

FIG. 16 is a schematic sectional side view of an article, according to another embodiment of the present disclosure;

FIG. 17A is a schematic top view of an article, according to another embodiment of the present disclosure;

FIG. 17B is a schematic sectional view of the article taken along a section line C-C′ shown in FIG. 17A, according to an embodiment of the present disclosure;

FIG. 18 is a schematic top view of a portion of an article, according to an embodiment of the present disclosure; and

FIG. 19 is a flow chart illustrating a method of using the article, according to an embodiment of the present disclosure.

### DETAILED DESCRIPTION

In the following description, reference is made to the accompanying figures that form a part thereof and in which various embodiments are shown by way of illustration. It is to be understood that other embodiments are contemplated and may be made without departing from the scope or spirit of the present disclosure. The following detailed description, therefore, is not to be taken in a limiting sense.

As used herein, the term “adhesive” generally refers to polymeric compositions useful to adhere together two adherends. Examples of adhesive may include curable adhesives, heat activated adhesives, pressure sensitive adhesives, or combinations thereof.

As used herein, the term “layer” generally refers to a thickness of material within a film that has a relatively consistent formula. The layers may be of any type of material including polymeric, cellulosic, and metallic, or a blend thereof. A given polymeric layer may be of a single polymer-type or may include a blend of polymers and may be accompanied by additives. A given layer may be combined or connected to other layers to form films. A layer may be either partially or fully continuous as compared to adjacent layers. A given layer may be partially or fully coextensive with adjacent layers. A layer may contain sub-layers.

As used herein, all numbers should be considered modified by the term “about”. As used herein, “a,” “an,” “the,” “at least one,” and “one or more” are used interchangeably.

As used herein as a modifier to a property or attribute, the term “generally”, unless otherwise specifically defined, means that the property or attribute would be readily recognizable by a person of ordinary skill but without requiring absolute precision or a perfect match (e.g., within +/−20% for quantifiable properties).

The term “substantially”, unless otherwise specifically defined, means to a high degree of approximation (e.g., within +/−10% for quantifiable properties) but again without requiring absolute precision or a perfect match.

The term “about”, unless otherwise specifically defined, means to a high degree of approximation (e.g., within +/−5% for quantifiable properties) but again without requiring absolute precision or a perfect match.

As used herein, the terms “first” and “second” are used as identifiers. Therefore, such terms should not be construed as limiting of this disclosure. The terms “first” and “second” when used in conjunction with a feature or an element can be interchanged throughout the embodiments of this disclosure.

As used herein, “at least one of A and B” should be understood to mean “only A, only B, or both A and B”.

As used herein, the recitations of numerical ranges by endpoints include all numbers subsumed within that range as well as the endpoints (e.g., 1 to 5 includes 1, 1.5, 2, 2.75, 3, 3.80, 4, 5, etc.).

According to aspects of this disclosure, an article includes at least one adhesive layer including an adhesive. The article further includes at least one shell enclosing the at least one adhesive layer. The at least one shell incudes a main member including a first major surface and a second major surface opposite to the first major surface. The second major surface is coupled to the at least one adhesive layer. The main member further includes a perimeter disposed between the first major surface and the second major surface. The at least one shell further includes a side member extending from and coupled to at least a portion of the perimeter of the main member. The side member extends from the perimeter beyond the second major surface of the main member and at least partially surrounds the at least one adhesive layer. The side member is configured to flex relative to the main member.

In the article of the present disclosure, the at least one shell may enclose the at least one adhesive layer on an external surface to which the article is attached through the main member and the side member. Specifically, the side member may at least partially surround the at least one adhesive layer and may cover edges or a perimeter of the at least one adhesive layer. Thus, the at least one shell may protect the at least one adhesive layer from environmental factors, such as dirt, moisture, etc.

Moreover, the at least one shell may also protect the edges or the perimeter of the at least one adhesive layer from catching on external objects, such as clothing, thereby preventing the at least one adhesive layer from being peeled up from an external surface (e.g., skin of a user) to which the article may be attached. Further, the at least one shell may also distribute lifting forces over a larger region, thereby mitigating chances of edge lifting. Longer attachment period may improve a user experience and may enable less landfill waste.

Additionally, as compared to the conventional articles, the side member of the present disclosure may flex relative to the main member. Thus, the at least one shell may also protect the at least one adhesive layer on external surfaces that may be flexible and/or curved by allowing the flexible side member to at least partially surround the at least one adhesive layer at all times. In other words, having the flexible side member extending from and coupled to at least the portion of the perimeter of the main member (which may be relatively rigid) may protect the at least one adhesive layer even when the article is attached to the external surfaces that may be flexible and/or curved. Thus, the article of the present disclosure may provide a highly compliant attachment with longer attachment period and a better quality of life for the user.

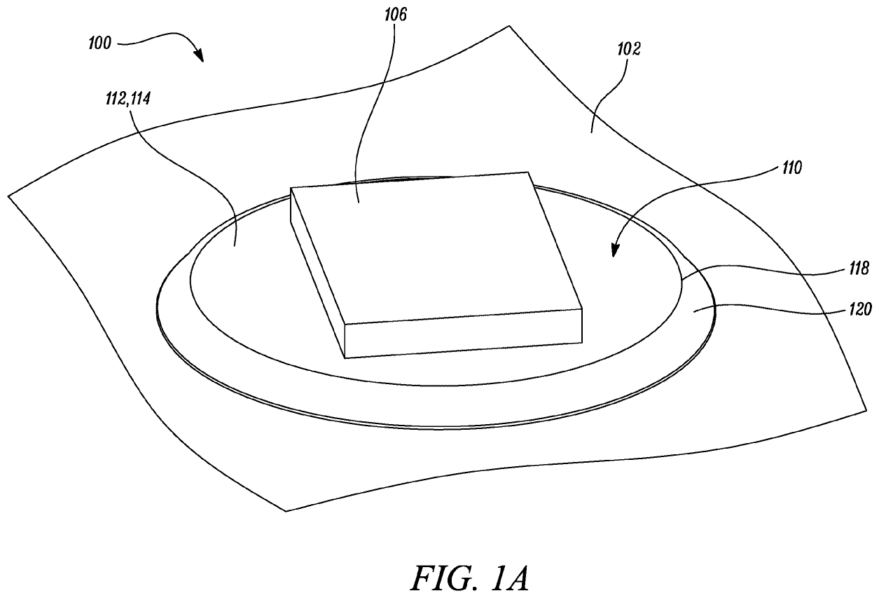

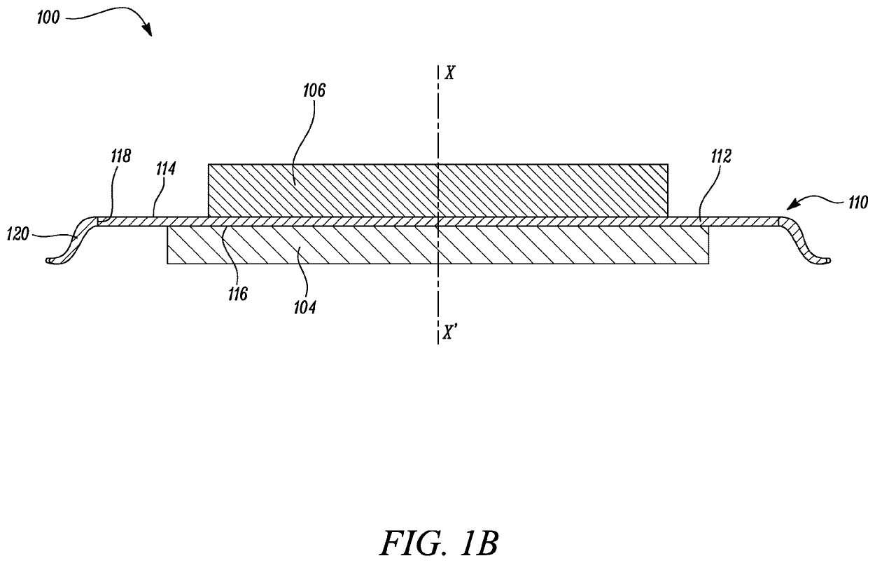

Referring now to Figures, FIG. 1A is a schematic perspective view of an article 100. FIG. 1B is a schematic sectional side view of the article 100. Referring now to FIGS. 1A and 1B, the article 100 includes at least one adhesive layer 104 (shown in FIG. 1B) including an adhesive. In some examples, the article 100 further includes at least one shell 110 enclosing the at least one adhesive layer 104. The term “at least one adhesive layer 104” is interchangeably referred to hereinafter as “the adhesive layer 104”. The term “at least one shell 110” is interchangeably referred to hereinafter as “the shell 110”.

As shown in FIG. 1B, the at least one shell 110 includes a main member 112. The main member 112 includes a first major surface 114 and a second major surface 116 opposite to the first major surface 114. In some examples, the first major surface 114 and the second major surface 116 are substantially planar and parallel to each other. The second major surface 116 is coupled to the at least one adhesive layer 104.

In some examples, the article 100 is configured to be attached to an external surface 102 (shown in FIG. 1A) via the adhesive of the at least one adhesive layer 104. In some examples, the adhesive is a pressure sensitive adhesive or a stretch-release adhesive. In some examples, the article 100 may further include a release liner (not shown) releasably attached to the adhesive layer 104. The release liner may be removed prior to bonding of the adhesive layer 104 with the external surface 102.

In the illustrated embodiment of FIGS. 1A and 1B, the main member 112 is circular. However, in some other embodiments, the main member 112 is one of rectangular, square, elliptical, oval, and polygonal. It should be understood that the main member 112 may be of any shape and geometry based on application requirements, including irregular shapes. Further, the external surface 102 is shown in FIG. 1A as a planar surface. However, the external surface 102 may also be curved or irregular.

The main member 112 further includes a perimeter 118 disposed between the first major surface 114 and the second major surface 116. The at least one shell 110 further includes a side member 120 extending from and coupled to at least a portion of the perimeter 118 of the main member 112. The side member 120 extends from the perimeter 118 beyond the second major surface 116 of the main member 112 and at least partially surrounds the at least one adhesive layer 104. In some examples, the side member 120 further extends towards the at least one adhesive layer 104 such that the side member 120 is inclined obliquely with respect to a normal X-X′ to the first major surface 114 and the second major surface 116.

In some examples, the main member 112 and the side member 120 are integrally formed. In some examples, the side member 120 may prevent environmental agents, such as dirt and moisture, from reaching the adhesive layer 104. In some examples, the side member 120 and the main member 112 are made from the same material. The side member 120 is configured to flex relative to the main member 112. In some examples, the side member 120 is hingedly coupled to at least the portion of the perimeter 118 of the main member 112. Thus, the at least one shell 110 may protect the at least one adhesive layer 104 on the external surface 102 that may be flexible and/or curved by allowing the side member 120 to at least partially surround the at least one adhesive layer 104 at all times.

In some examples, the shell 110 may be a part of a medical diagnostic device and the external surface 102 may be the skin of a user. In some examples, the article 100 further includes a sensor 106 disposed on the first major surface 114 of the main member 112. In some examples, the sensor 106 may collect physiologic data from the skin of the user. For examples, the sensor may collect information relating to one or more of calories burned, steps taken, activity levels, sleep patterns, and other parameters.

In some examples, the sensor 106 may collect one or more of the following data: skin surface temperature data of the user, core body temperature data of the user, motion or inactivity data of the user, body angle data of the user, heart rate and/or ECG data of the user, and respiration information of the user. In some examples, the sensor 106 may utilize software algorithms to process the collected data and determine or provide indication of one or more of sleep quality and quantity, activity level, step count, caloric expenditures, etc.

In some examples, the sensor 106 may include multiple sensors and/or multiple sensor types that collect data. In some examples, the sensor 106 may include provisions that enable wireless transfer of collected data to an external device, e.g., a computer or a mobile device. In some examples, the sensor 106 may, continuously or intermittently, transmit the collected data wirelessly to the external device, such that the collected data may be viewed or displayed in real time on the external device. In some examples, the sensor 106 may also utilize cloud-based infrastructure for storage and presentation of the collected and/or processed data.

In some examples, the article 100 or the one or more associated components may be in the form of, for example, a sheet, a film, or any similar construction. In some examples, the article 100 or the one or more associated components may be made of plastic, woven and non-woven materials, and/or fabrics. In some examples, the article 100 or the one or more associated components may be made of elastic materials (including rubber, such as natural rubber, synthetic rubber, nitrile rubber, silicone rubber, urethane rubbers, chloroprene rubber, ethylene vinyl acetate or EVA rubber, etc.), inelastic materials (including polyethylene and polycarbonate), polyesters, acrylics, polysulphones, thermoplastic materials, thermoset materials, and biodegradable polymers.

Examples of thermoplastic materials may include, but are not limited to, one or more of polyolefins (e.g., polyethylene, such as high density polyethylene or HDPE, medium density polyethylene or MDPE, low density polyethylene or LDPE, linear low density polyethylene or LLDPE, metallocene polyethylene, and the like, and combinations thereof), polypropylene (e.g., atactic and syndiotactic polypropylene), polyamides (e.g., nylon), polyurethane, polyacetal (e.g., Delrin®), polyacrylates, and polyesters (such as polyethylene terephthalate or PET, polyethylene terephthalate glycol or PETG, and aliphatic polyesters such as polylactic acid), fluoroplastics (such as THV from 3M company, St. Paul, MN), and combinations thereof.

Examples of thermoset materials may include, but are not limited to, one or more of polyurethanes, silicones, epoxies, melamine, phenol-formaldehyde resin, and combinations thereof. Examples of biodegradable polymers may include, but are not limited to, one or more of polylactic acid (PLA), poly glycolic acid (PGA), poly(caprolactone), copolymers of lactide and glycolide, poly (ethylene succinate), polyhydroxybutyrate, and combinations thereof.

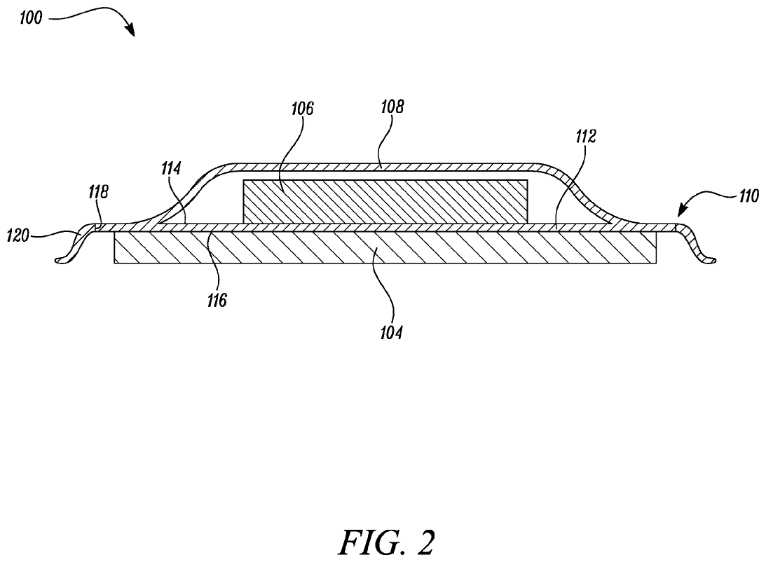

FIG. 2 is a schematic sectional side view of the article 100, according to another embodiment of the present disclosure. In the illustrated embodiment of FIG. 2, the at least one shell 110 further includes a housing portion 108 coupled to the main member 112 and extending from the first major surface 114 opposite to the at least one adhesive layer 104. In some examples, the housing portion 108 encloses the sensor 106. Thus, the housing portion 108 may protect the sensor 106 from environmental factors and external objects.

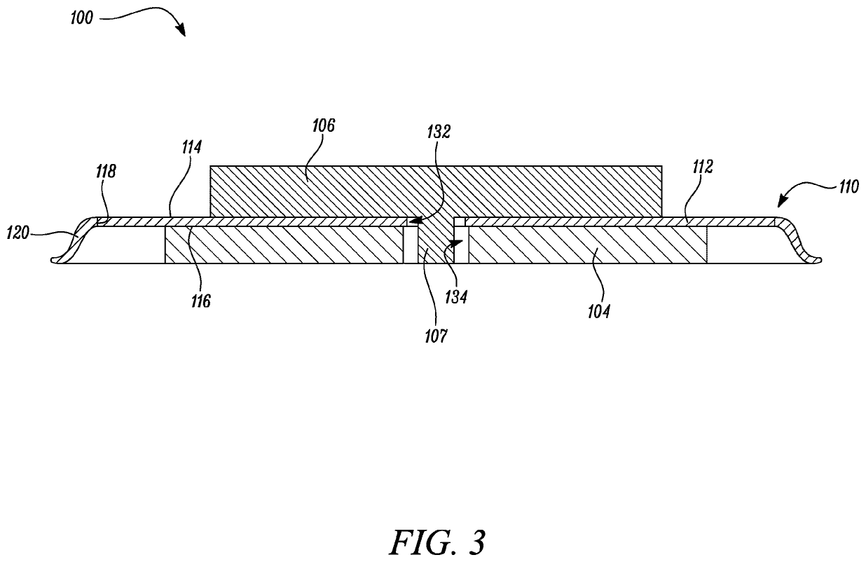

FIG. 3 is a schematic sectional side view of the article 100, according to yet another embodiment of the present disclosure. In the illustrated embodiment of FIG. 3, the main member 112 further includes a first aperture 132 extending through the main member 112 between the first major surface 114 and the second major surface 116. In some examples, the at least one adhesive layer 104 includes a second aperture 134 extending therethrough and at least partially aligned with the first aperture 132. In some examples, the sensor 106 further includes a sensing portion 107 extending at least partially through the first aperture 132 and the second aperture 134. In some examples, the sensing portion 107 may contact the skin of the user for collecting physiologic data from the skin of the user.

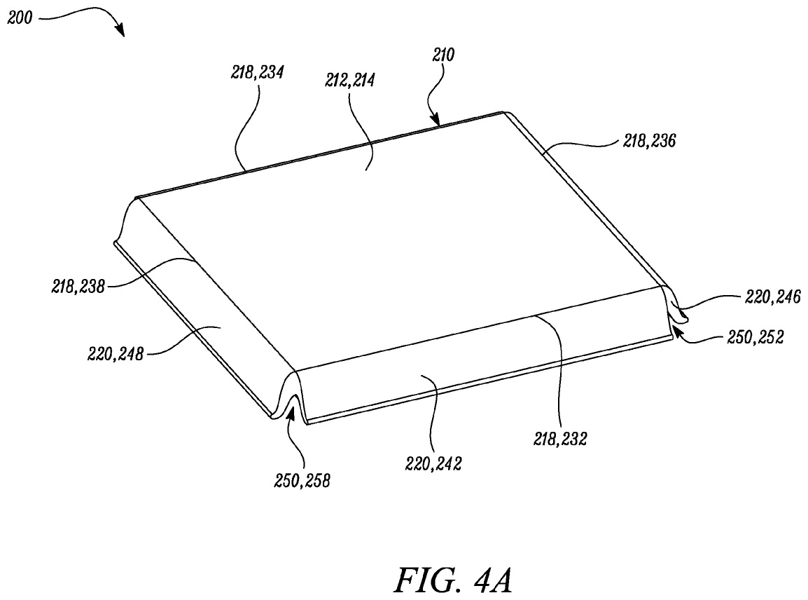

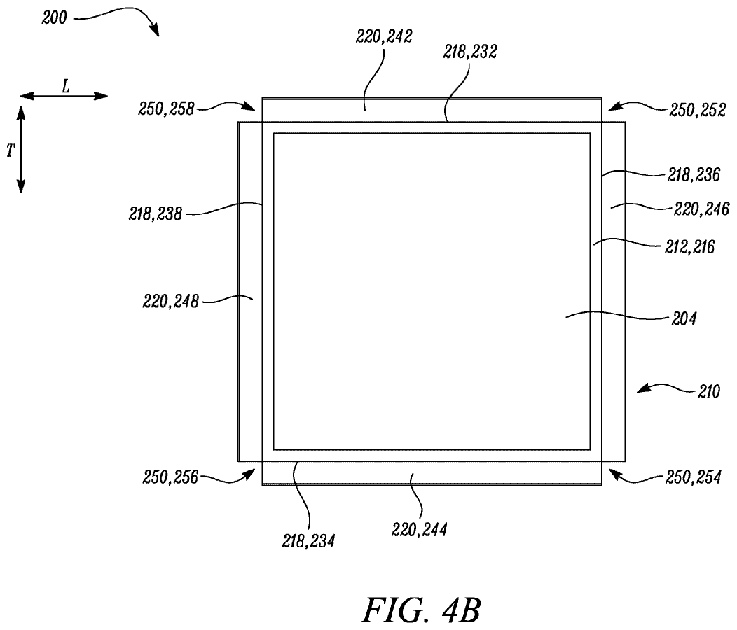

FIG. 4A is a schematic perspective view of an article 200. In some examples, the article 200 may be functionally similar to the article 100 of FIGS. 1A-3. FIG. 4B is a schematic bottom view of the article 200. Referring now to FIGS. 4A and 4B, the article 200 includes at least one adhesive layer 204 (shown in FIG. 4B). The article 200 further includes at least one shell 210 enclosing the at least one adhesive layer 204. The at least one shell 210 includes a main member 212 including a first major surface 214 (shown in FIG. 4A), a second major surface 216 (shown in FIG. 4B) opposite to the first major surface 214, and a perimeter 218 disposed between the first major surface 214 and the second major surface 216. The second major surface 216 is coupled to the at least one adhesive layer 204.

As shown in FIG. 4B, in some examples, the perimeter 218 includes a first longitudinal edge 232 extending along a longitudinal axis L, a second longitudinal edge 234 opposing the first longitudinal edge 232 and extending along the longitudinal axis L, a first transverse edge 236 extending between the first longitudinal edge 232 and the second longitudinal edge 234 along a transverse axis T orthogonal to the longitudinal axis L, and a second transverse edge 238 opposing the first transverse edge 236 and extending between the first longitudinal edge 232 and the second longitudinal edge 234 along the transverse axis T. In the illustrated embodiment of FIG. 4A, the main member 212 is rectangular. However, it should be understood that the main member 212 may be of any shape (e.g., circular, polygonal, elliptical, irregular, etc.) and may include any number of sides (e.g., a poly gon with three or more sides).

The at least one shell 210 further includes a side member 220 extending from and coupled to at least a portion of the perimeter 218 of the main member 212. In some examples, the side member 220 and the main member 212 are made from the same material. In some examples, the side member 220 includes a first side portion 242 extending from the first longitudinal edge 232, a second side potion 244 extending from the second longitudinal edge 234, a third side portion 246 extending from the first transverse edge 236, and a fourth side portion 248 extending from the second transverse edge 238. In some examples, the first side portion 242, the second side portion 244, the third side portion 246, and the fourth side portion 248 together at least partially surround the at least one adhesive layer 204. In some examples, the first, second, third and fourth side portions 242, 244, 246, 248 are similar to each other. Each of the first, second, third and fourth side portions 242, 244, 246, 248 is hingedly coupled to and configured to flex relative to the main member 212.

In some examples, the side member 220 includes one or more cutouts 250 extending at least partially therethrough. In some examples, the side member 220 further defines a first cutout 252 between the first side portion 242 and the third side portion 246, a second cutout 254 between the second side portion 244 and the third side portion 246, a third cutout 256 between the second side portion 244 and the fourth side portion 248, and a fourth cutout 258 between fourth side portion 248 and the first side portion 242, such that the first, second, third, and fourth side portions 242, 244, 246, 248 are spaced apart from each other. In some examples, the first, second, third and fourth cutouts 252, 254, 256, 258 are similar to each other.

In the illustrated embodiment of FIGS. 4A and 4B, the side member 220 includes four cutouts 250. However, it should be understood that the side member 220 may include any number of cutouts 250. Further, in alternative embodiments, the side member 220 may be devoid of any cutouts.

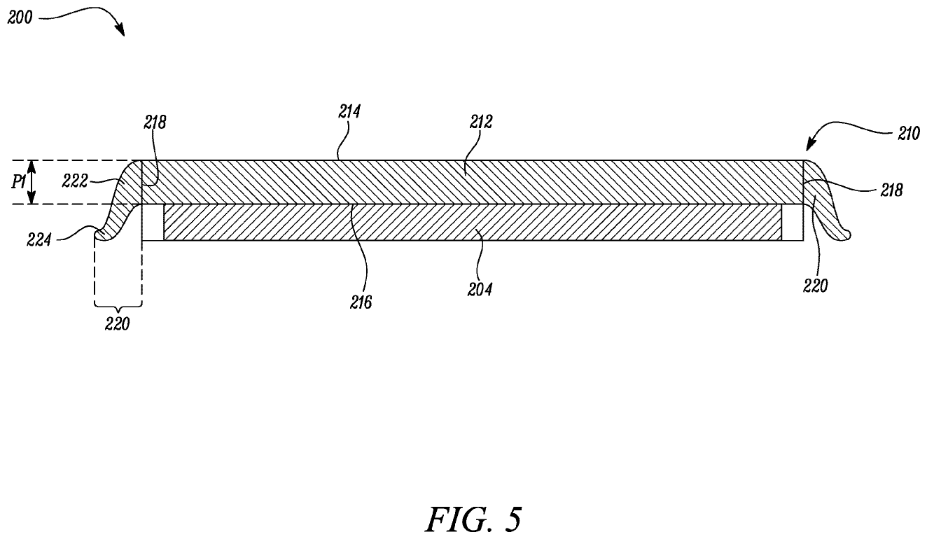

FIG. 5 is a schematic sectional side view of the article 200. In some examples, the side member 220 includes a side main portion 222 extending from the perimeter 218 and a curved lip 224 extending from the side main portion 222 opposite to the perimeter 218. It should be understood that each of the first, second, third and fourth side portions 242, 244, 246, 248 (shown in FIGS. 4A and 4B) includes the side main portion 222 and the curved lip 224. In some examples, the curved lip 224 may engage with the external surface 102 (shown in FIG. 1A), thereby enabling the at least one shell 210 to enclose the at least one adhesive layer 204.

In some examples, the side main portion 222 is hingedly coupled to the perimeter 218. Further, in some examples, the side main portion 222 forms a living hinge. Thus, the side main portion 222 may be able to flex relative to the main member 212. In some examples, the side member 220 tapers away from the perimeter 218. This may enhance a flexibility of the side member 220 with respect to the main member 212. Specifically, the side main portion 222 tapers away from the perimeter 218 to the curved lip 224, such that the side main portion 222 has a maximum thickness P1 adjacent to the perimeter 218.

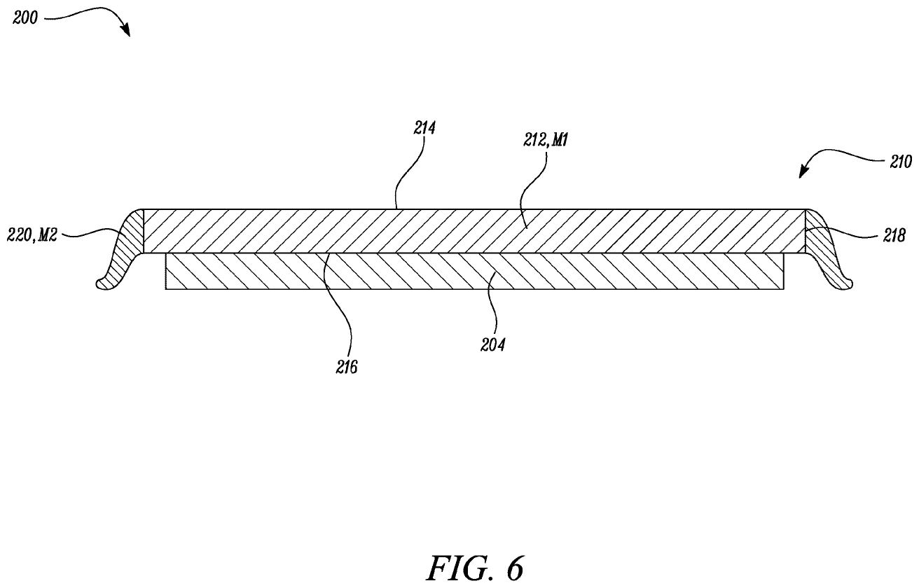

FIG. 6 is a schematic sectional side view of the article 200, according to another embodiment of the present disclosure. In the illustrated embodiment of FIG. 6, the main member 212 is made from a first material M1 and the side member 220 is made from a second material M2 different from the first material M1. In some examples, an elastic modulus of the first material M1 is greater than an elastic modulus of the second material M2 by a factor of at least two. In some examples, the elastic modulus of the first material M1 is greater than the elastic modulus of the second material M2 by a factor of at least three, at least four, or at least five. Thus, the side member 220 may flex relative to the main member 212 by virtue of its inherent material properties.

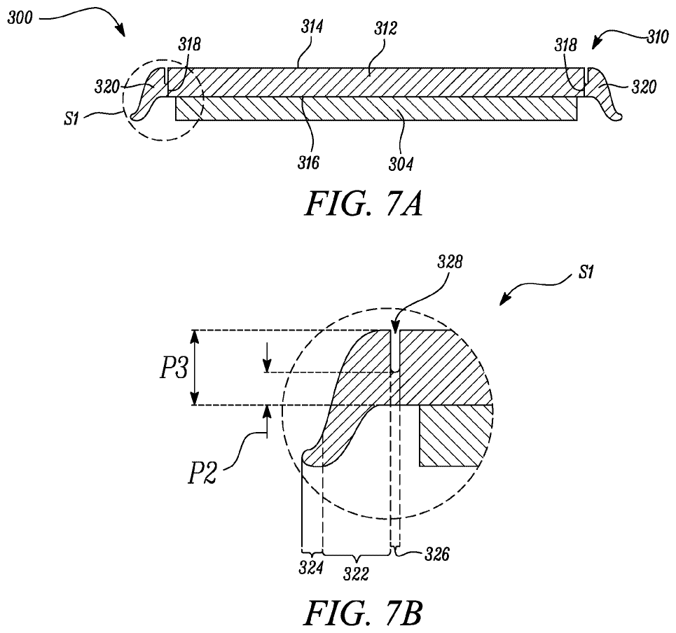

FIG. 7A is a schematic sectional side view of an article 300. In some examples, the article 300 may be functionally similar to the article 100 of FIGS. 1A-3 and article 200 of FIGS. 4A-6. The article 300 includes at least one adhesive layer 304. The article 300 further includes at least one shell 310 enclosing the at least one adhesive layer 304. The at least one shell 310 includes a main member 312 including a first major surface 314, a second major surface 316 opposite to the first major surface 314, and a perimeter 318 disposed between the first major surface 314 and the second major surface 316. The second major surface 316 is coupled to the at least one adhesive layer 304. The at least one shell 310 further includes a side member 320 extending from and coupled to at least a portion of the perimeter 318 of the main member 312.

FIG. 7B is an enlarged schematic view of a section S1 (shown in FIG. 7A) of the article 300. As shown in FIG. 7B, in some examples, the side member 320 includes a side narrow portion 326 disposed adjacent to the perimeter 318 and hingedly coupled to the perimeter 318. In some examples, the side member 320 further includes a side main portion 322 extending from the side narrow portion 326.

In some examples, a thickness P2 of the side narrow portion 326 is less than a maximum thickness P3 of the side main portion 322. In some examples, the side main portion 322 tapers away from the side narrow portion 326, such that the maximum thickness P3 of the side main portion 322 is adjacent to the side narrow portion 326. In some examples, the side member 320 further includes a curved lip 324 extending from the side main portion 322 opposite to the perimeter 318. In some examples, the curved lip 324 may engage with the external surface 102 (shown in FIG. 1A), thereby enabling the at least one shell 310 to enclose the at least one adhesive layer 304.

In some examples, the side narrow portion 326 includes at least one notch 328 extending partially therethrough. In the illustrated embodiments of FIGS. 7A and 7B, the at least one notch 328 is a single notch extending from the side narrow portion 326 towards the first major surface 314. The side narrow portion 326 forms a living hinge. Thus, the side narrow portion 326 may allow the side member 320 to flex relative to the main member 312, thereby at least partially surrounding the at least one adhesive layer 304 at all times even on external surfaces that may be flexible and/or curved.

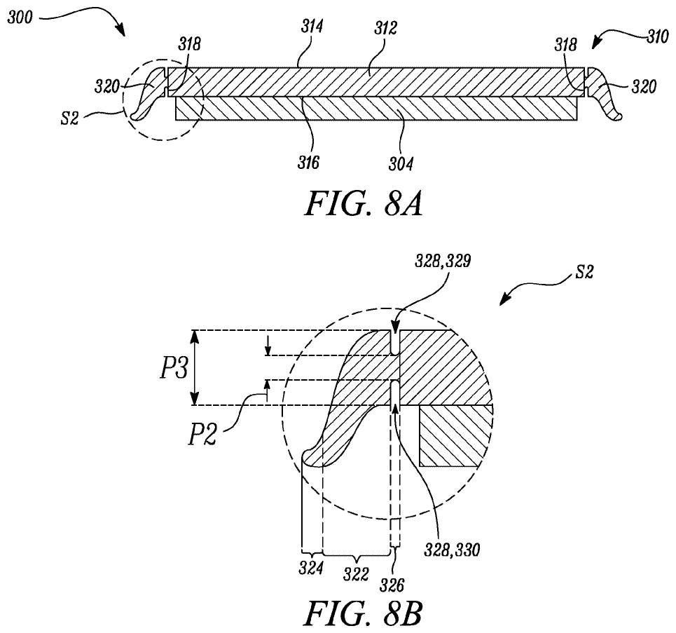

FIG. 8A is a schematic sectional side view of the article 300, according to another embodiment of the present disclosure. FIG. 8B is an enlarged schematic view of a section S2 (shown in FIG. 8A) of the article 300. As shown in FIG. 8B, in some examples, the at least one notch 328 includes a first notch 329 disposed proximal to the first major surface 314 of the main member 312 and a second notch 330 aligned with and extending towards the first notch 329. In some examples, the second notch 330 is disposed distal to the first major surface 314 of the main member 312.

In some examples, the first notch 329 and the second notch 330 may be similar to each other. In the illustrated embodiments of FIGS. 8A and 8B, the first notch 329 extends from the side narrow portion 326 towards the first major surface 314, while the second notch 330 extends from the side narrow portion 326 towards the second major surface 316. The first notch 329 and the second notch 330 may enhance the flexibility of the side member 320 with respect to the main member 312.

In some examples, the thickness P2 of the side narrow portion 326 is less than the maximum thickness P3 of the side main portion 322. In some examples, the side main portion 322 tapers away from the side narrow portion 326, such that the maximum thickness P3 of the side main portion 322 is adjacent to the side narrow portion 326. In some examples, the side member 320 further includes the curved lip 324 extending from the side main portion 322 opposite to the perimeter 318. In some examples, the curved lip 324 may engage with the external surface 102 (shown in FIG. 1A), thereby enabling the at least one shell 310 to enclose the at least one adhesive layer 304.

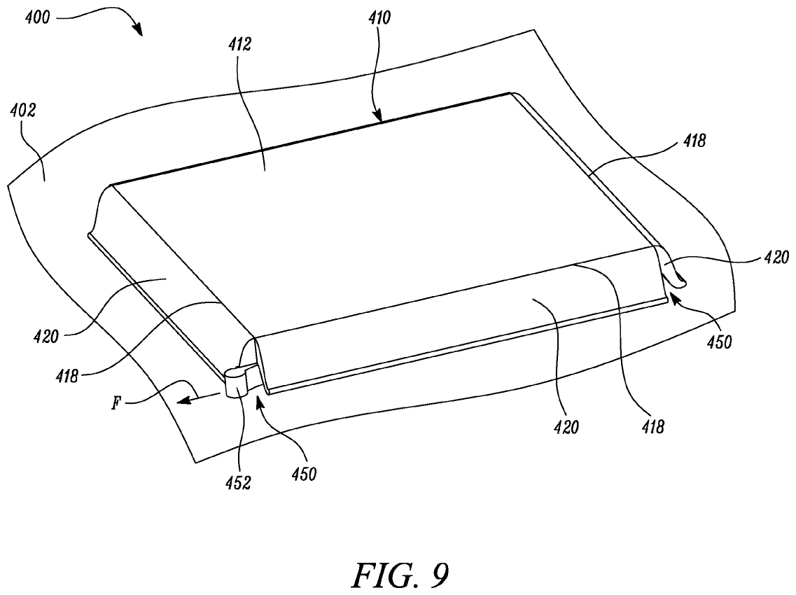

FIG. 9 is a schematic perspective view of an article 400. The article 400 may be functionally similar to the article 100 of FIGS. 1A-3, the article 200 of FIGS. 4A-6, and the article 300 of FIGS. 7-8. The article 400 further includes at least one shell 410 enclosing at least one adhesive layer (e.g., the adhesive layer 204 shown in FIG. 4B). The shell 410 includes a main member 412 including a perimeter 418 and a side member 420 extending from and coupled to at least a portion of the perimeter 418 of the main member 412. In some examples, the article 400 is configured to be attached to an external surface 402 via the adhesive layer.

In some examples, the side member 420 includes one or more cutouts 450 extending at least partially therethrough. In some examples, the article 400 further includes at least one tab 452 connected to and extending from the adhesive layer. In some examples, the at least one tab 452 extends through one of the one or more cutouts 450 and is at least partially disposed outside of the shell 410. In some examples, the at least one tab 452 may receive a pull force F from a user of the article 400, allowing the adhesive layer to be pulled, thereby releasing the adhesive layer from each of the shell 410 and the external surface 402.

In some examples, the at least one tab 452 may be made from a different material as that of the adhesive layer. In some other examples, the at least one tab 452 may be made from the same material as that of the adhesive layer. In some examples, upon application of the pull force F by the user on the at least one tab 452, the adhesive layer may slide out from the one of the one or more cutouts 450, thereby releasing the shell 410 from the external surface 402. Alternatively, upon application of the pull force F by the user on the at least one tab 452, the adhesive layer may slide out underneath the side member 420, thereby releasing the shell 410 from the external surface 402.

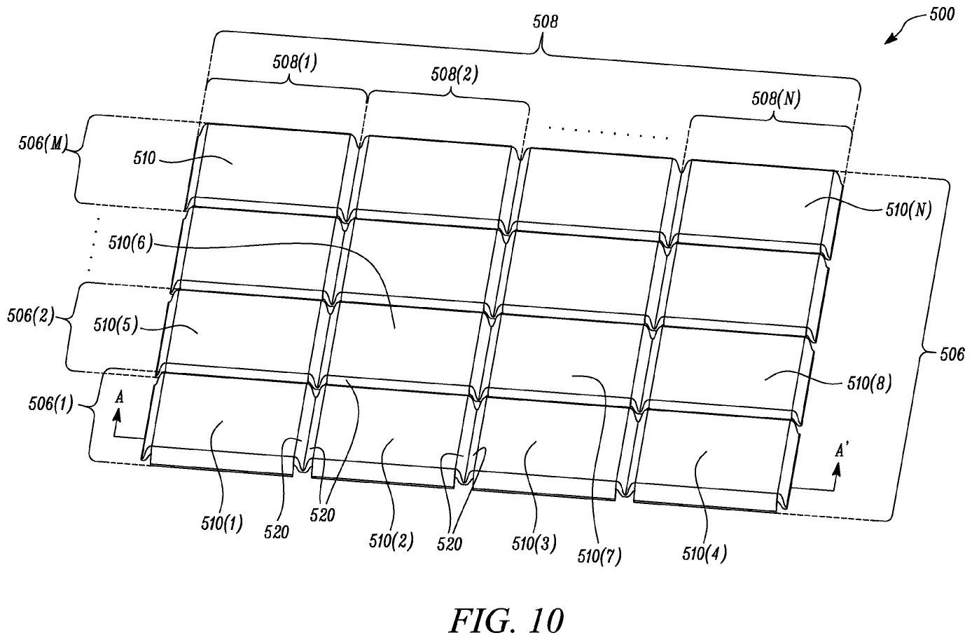

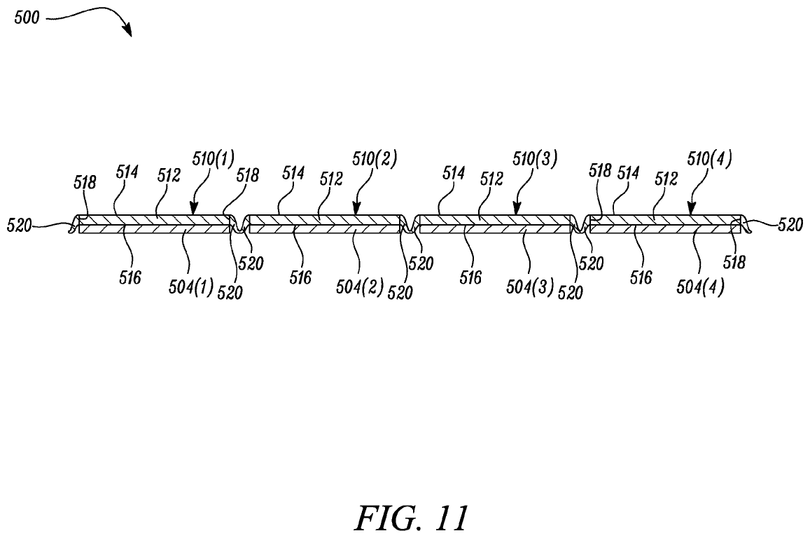

FIG. 10 is a schematic perspective view of an article 500. In some examples, the article 500 may be functionally similar to the article 100 of FIGS. 1A-3, the article 200 of FIGS. 4A-6, and the article 300 of FIGS. 7-8. FIG. 11 is a schematic cross-sectional view of the article 500 taken along a section line A-A′ shown in FIG. 10. Referring now to FIGS. 10 and 11, the article 500 includes at least one adhesive layer 504 (shown in FIG. 11). The article 500 further includes at least one shell 510 enclosing the at least one adhesive layer 504.

In the illustrated embodiment of FIGS. 10 and 11, the at least one shell 510 includes a plurality of shells 510(1), 510(2), . . . , 510(P) (collectively, shells 510) connected to each other, where P is an integer corresponding to a total number of shells (e.g., P=10, 20, etc.). In the illustrated embodiment of FIG. 10, P=16. In some examples, the plurality of shells 510 are arranged in an array including a plurality of rows 506(1), 506(2), . . . , 506(M) (collectively, rows 506), where M is an integer corresponding to a total number of rows (e.g., M=10, 20, etc.) and a plurality of columns 508(1), 508(2), . . . , 508(N) (collectively, columns 508), where N is an integer corresponding to a total number of columns (e.g., N=10, 20, etc.). In the illustrated embodiment of FIG. 10, M=4 and N=4.

As shown in FIG. 11, each of the plurality of shells 510(1)-510(P) includes a main member 512 including a first major surface 514, a second major surface 516 opposite to the first major surface 514, and a perimeter 518 disposed between the first major surface 514 and the second major surface 516. In the illustrated embodiment of FIGS. 10 and 11, the main member 512 is substantially rectangular.

However, it should be understood that the main member 512 may be of any shape (e.g., circular, poly gonal, elliptical, irregular, etc.) and may include any number of sides (e.g., a polygon with three or more sides). In some examples, the at least one adhesive layer 504 includes a plurality of adhesive layers 504(1), 504(2), . . . , 504(P) (collectively, adhesive layers 504) corresponding to the plurality of shells 510 and spaced apart from each other.

In some examples, each adhesive layer 504(1)-504(P) from the plurality of adhesive layers 504 is coupled to the second major surface 516 of the main member 512 of a corresponding shell 510(1)-510(P) from the plurality of shells 510. For example, the adhesive layer 504(1) is coupled to the second major surface 516 of the main member 512 of the shell 510(1), the adhesive layer 504(2) is coupled to the second major surface 516 of the main member 512 of the shell 510(2), and so on.

Each of the plurality of shells 510(1)-510(P) further includes a side member 520 extending from and coupled to at least a portion of the perimeter 518 of the main member 512. In some examples, the side member 520 of each of the plurality of shells 510(1)-510(P) is at least partially coupled to the side member 520 of at least one adjacent shell 510(1)-510(P) from the plurality of shells 510. For example, the side member 520 of the shell 510(1) is coupled to the side member 520 of the shell 510(2).

Referring again to FIG. 10, in some examples, the side member 520 of each of the plurality of shells 510(1)-510(P) disposed in a corresponding row 506(1)-506(M) from the plurality of rows 506 and a corresponding column 508(1)-508(N) from the plurality of columns 508 is coupled to the side member 520 of each adjacent shell 510(1)-510(P) disposed in the corresponding row 506(1)-506(M) and each adjacent shell 510(1)-510(P) disposed in the corresponding column 508(1)-508(N). For example, the shell 510(2) disposed in the row 506(1) and the column 508(2) is connected to the adjacent shells 510(1), 510(3) disposed in the row 506(1) and the adjacent shell 510(6) disposed in the column 508(2).

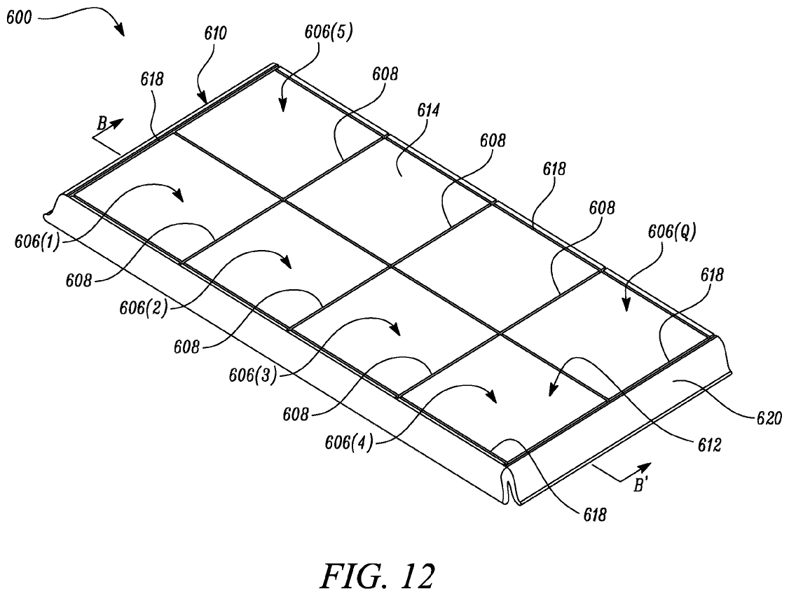

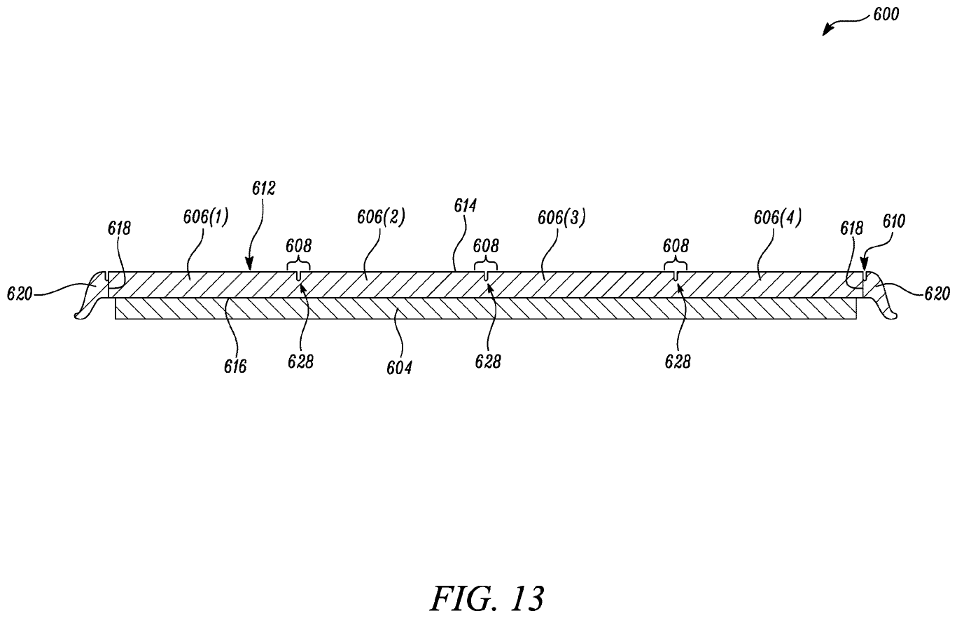

FIG. 12 is a schematic perspective view of an article 600. In some examples, the article 600 may be functionally similar to the article 500 of FIGS. 10 and 11. FIG. 13 is a schematic sectional view of the article 600 taken along a section line B-B′ shown in FIG. 12. Referring now to FIGS. 12 and 13, the article 600 includes at least one adhesive layer 604 (shown in FIG. 13). The article 600 further includes at least one shell 610 enclosing the at least one adhesive layer 604.

In the illustrated embodiment of FIGS. 12 and 13, the at least one shell 610 includes a single shell 610. In some examples, the single shell 610 includes a main member 612 including a first major surface 614, a second major surface 616 (shown in FIG. 13) opposite to the first major surface 614, and a perimeter 618 disposed between the first major surface 614 and the second major surface 616. The at least one shell 610 further includes a side member 620 extending from and coupled to at least a portion of the perimeter 618 of the main member 612.

In some examples, the main member 612 of the single shell 610 includes a plurality of main segments 606(1), 606(2), . . . , 606(Q) (collectively, main segments 606) connected to each other, where Q is an integer corresponding to a total number of main segments (e.g., Q=10, 20, etc.). In the illustrated embodiment of FIG. 12, Q=8. In some examples, the plurality of main segments 606 together define the perimeter 618. In the illustrated embodiment of FIGS. 12 and 13, the plurality of main segments 606 are substantially rectangular. However, it should be understood that the plurality of main segments 606 may be of any shape (e.g., circular, polygonal, elliptical, irregular, etc.) and may include any number of sides (e.g., a polygon with three or more sides).

In some examples, the single shell 610 further includes a plurality of intermediate portions 608. In some examples, each of the plurality of intermediate portions 608 connects a corresponding adjacent pair of main segments 606(1)-606(Q) to each other. For example, the main segments 606(1) is connected to the main segment 606(2) via the corresponding intermediate portion 608. In some examples, the plurality of main segments 606 and the plurality of intermediate portions 608 together define the first major surface 614 and the second major surface 616 (shown in FIG. 13).

As shown in FIG. 13, in some examples, at least the second major surface 616 together defined by the plurality of main segments 606 and the plurality of intermediate portions 608 is planar. However, it should be understood that the first major surface 614 and the second major surface 616 may also be curved. In some examples, the at least one adhesive layer 604 includes a single adhesive layer 604 disposed on the second major surface 616 together defined by the plurality of main segments 606 and the plurality of intermediate portions 608. In some examples, each of the plurality of intermediate portions 608 includes at least one notch 628 extending partially therethrough. In the illustrated embodiment of FIG. 13, each intermediate portion 608 includes one notch 628 extending from the first major surface 614.

The at least one notch 628 may provide the flexibility to the main member 612, such that the plurality of main segments 606 may accommodate flexibility and curvature of an external surface to which the article 600 is attached while allowing the side member 620 to at least partially surround the single adhesive layer 604 at all times.

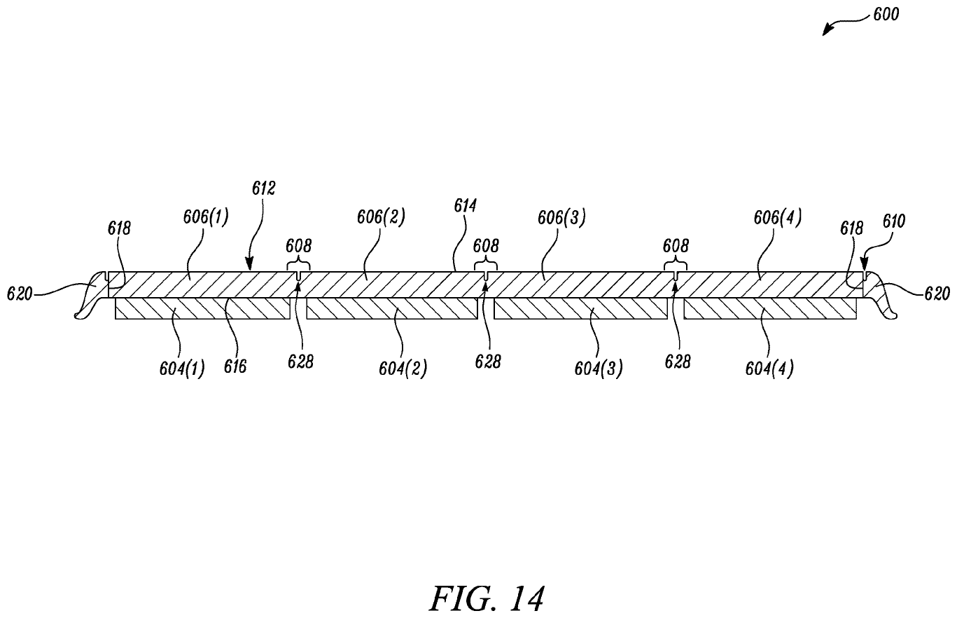

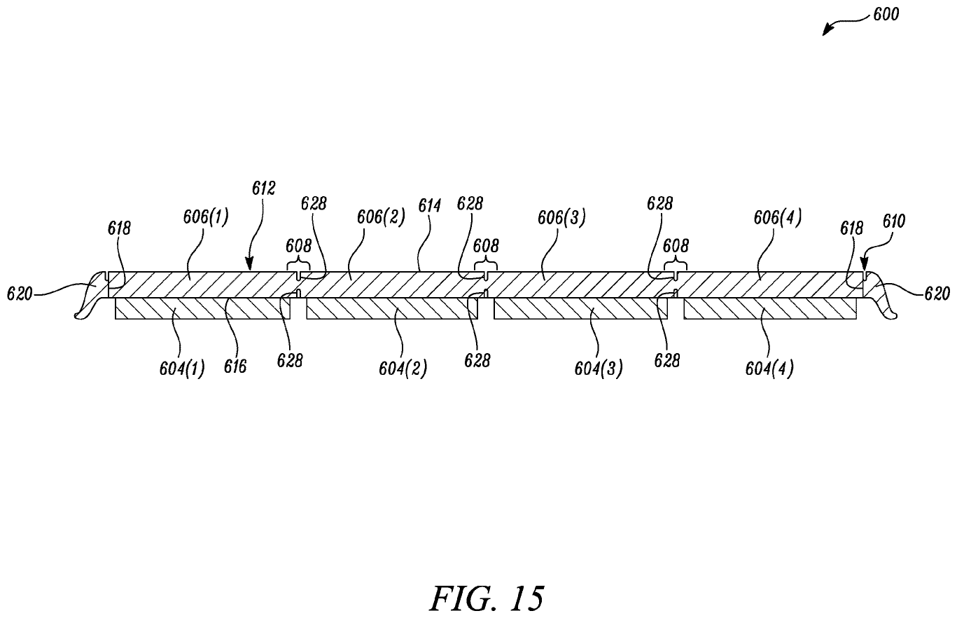

FIG. 14 is a schematic sectional view of the article 600 taken along the section line B-B′ shown in FIG. 12, according to another embodiment of the present disclosure. In the illustrated embodiment of FIG. 14, the at least one adhesive layer 604 includes a plurality of adhesive layers 604(1)-604(Q) (collectively, adhesive layers 604) corresponding to the plurality of main segments 606(1)-606(Q) and spaced apart from each other.

In some examples, each adhesive layer 604(1)-604(Q) from the plurality of adhesive layers 604 is coupled to a portion of the second major surface 616 of a corresponding main segment 606(1)-606(Q) from the plurality of main segments 606. For example, the adhesive layer 604(1) is coupled to a portion of the second major surface 616 of the corresponding main segment 606(1).

FIG. 15 is a schematic sectional view of the article 600 taken along the section line B-B′ shown in FIG. 12, according to another embodiment of the present disclosure. In the illustrated embodiment of FIG. 15, the at least one notch 628 includes a pair of notches 628 extending partially through each of the plurality of intermediate portions 608 and opposing each other. The pair of notches 628 may further enhance the flexibility of the main member 612. In the illustrated embodiment of FIG. 15, one of the pair of notches 628 extend from the first major surface 614, while the other of the pair of notches 628 extends from the second major surface 616.

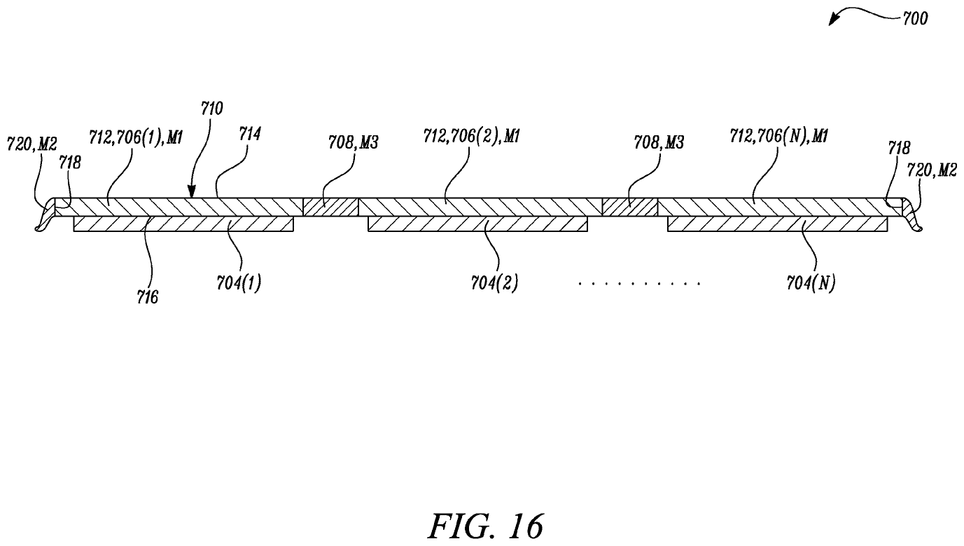

FIG. 16 is a schematic sectional view of an article 700. In some examples, the article 700 may be functionally similar to the article 600 of FIGS. 12-15. The article 700 includes at least one adhesive layer 704. The article 700 further includes at least one shell 710 enclosing the at least one adhesive layer 704.

In the illustrated embodiment of FIG. 16, the at least one shell 710 includes a single shell 710. In some examples, the single shell 710 includes a main member 712 including a first major surface 714, a second major surface 716 opposite to the first major surface 714, and a perimeter 718 disposed between the first major surface 714 and the second major surface 716. The at least one shell 710 further includes a side member 720 extending from and coupled to at least a portion of the perimeter 718 of the main member 712.

In some examples, the main member 712 of the single shell 710 includes a plurality of main segments 706(1), 706(2), . . . , 706(R) (collectively, main segments 706) connected to each other, where R is an integer corresponding to a total number of main segments (e.g., Q=10, 20, etc.). In some examples, the plurality of main segments 706 together define the perimeter 718.

In some examples, the single shell 710 further includes a plurality of intermediate portions 708. In some examples, each of the plurality of intermediate portions 708 connects a corresponding adjacent pair of main segments 706(1)-706(R) to each other. For example, the main segments 706(1) is connected to the main segment 706(2) via the intermediate portion 708. In some examples, the plurality of main segments 706 and the plurality of intermediate portions 708 together define the first major surface 714 and the second major surface 716.

In some examples, the at least one adhesive layer 704 includes a plurality of adhesive layers 704(1)-704(R) (collectively, adhesive layers 704) corresponding to the plurality of main segments 706(1)-706(R) and spaced apart from each other. In some examples, each adhesive layer 704(1)-704(R) from the plurality of adhesive layers 704 is coupled to a portion of the second major surface 716 of a corresponding main segment 706(1)-706(R) from the plurality of main segments 706. For example, the adhesive layer 704(1) is coupled to a portion of the second major surface 716 of the corresponding main segment 706(1).

In some examples, each of the plurality of main segments 706(1)-706(R) is made from the first material M1. In some examples, each of the plurality of intermediate portions 708 is made from a third material M3 different from the first material M1. In some examples, an elastic modulus of the first material M1 is greater than an elastic modulus of the third material M3 by a factor of at least two. In some examples, the elastic modulus of the first material M1 is greater than the elastic modulus of the third material M3 by a factor of at least three, at least four, or at least five. Thus, the plurality of intermediate portions 708 may enhance the flexibility of the main member 712 by virtue of its inherent material properties.

Additionally, the side member 720 is made from the second material M2 different from the first material M1. In some examples, the first material M1, the second material M2, and the third material M3 may be different from each other and may be selected based on application requirements.

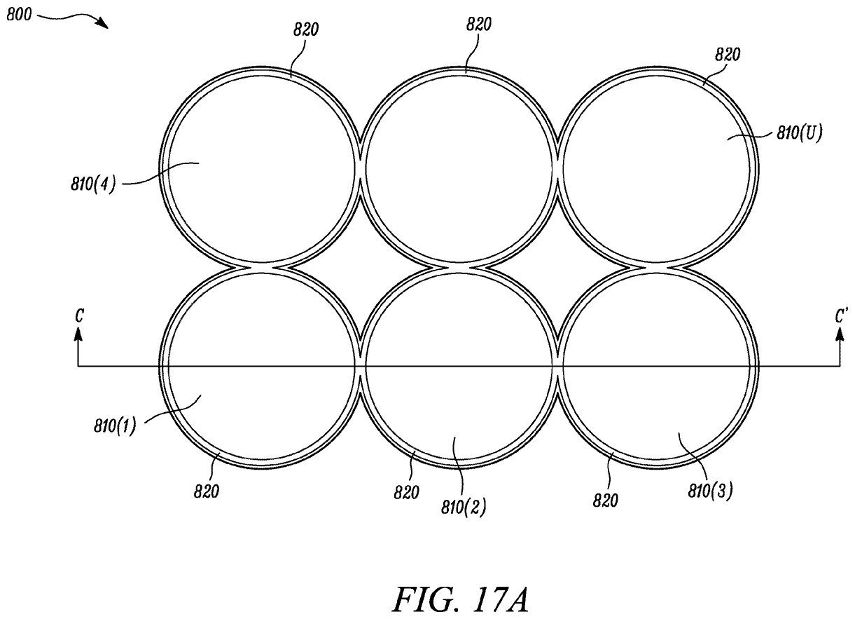

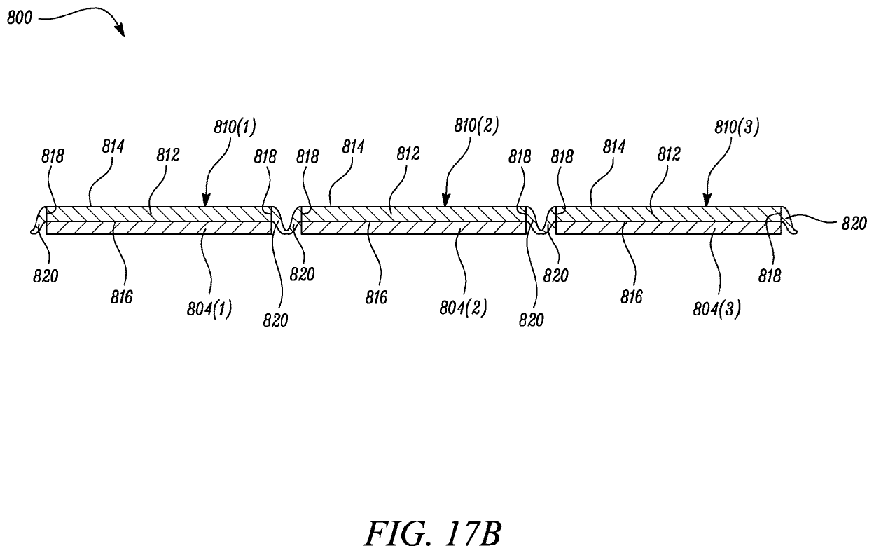

FIG. 17A is a schematic top view of an article 800. In some examples, the article 800 may be functionally similar to the article 500 of FIGS. 10 and 11. FIG. 17B is a schematic sectional view of the article 800 taken along a section line C-C′ shown in FIG. 17A. Referring now to FIGS. 17A and 17B, the article 800 includes at least one shell 810 enclosing at least one adhesive layer 804 (shown in FIG. 17B). In the illustrated embodiment of FIGS. 17A and 17B, the at least one shell 810 includes a plurality of shells 810(1), 810(2), . . . , 810(U) (collectively, shells 810) connected to each other, where U is an integer corresponding to a total number of shells (e.g., U=10, 20, etc.). In the illustrated embodiment of FIGS. 17A and 17B, U=6.

In some examples, each of the plurality of shells 810(1)-810(U) includes a main member 812 including a first major surface 814, a second major surface 816 opposite to the first major surface 814, and a perimeter 818 disposed between the first major surface 814 and the second major surface 816. In the illustrated embodiment of FIGS. 17A and 17B, the main member 812 is substantially circular.

However, it should be understood that the main member 812 may be of any shape based on application requirements.

In some examples, the at least one adhesive layer 804 includes a plurality of adhesive layers 804(1), 804(2), . . . , 804(U) (collectively, adhesive layers 804) corresponding to the plurality of shells 810 and spaced apart from each other. In some examples, each adhesive layer 804(1)-804(U) from the plurality of adhesive layers 804 is coupled to the second major surface 816 of the main member 812 of a corresponding shell 810(1)-810(U) from the plurality of shells 810. For example, the adhesive layer 804(1) is coupled to the second major surface 816 of the main member 812 of the shell 810(1), the adhesive layer 804(2) is coupled to the second major surface 816 of the main member 812 of the shell 810(2), and so on.

Each of the plurality of shells 810(1)-810(U) further includes a side member 820 extending from and coupled to at least a portion of the perimeter 818 of the main member 812. In some examples, the side member 820 of each of the plurality of shells 810(1)-810(U) is at least partially coupled to the side member 820 of at least one adjacent shell 810(1)-810(U) from the plurality of shells 810. For example, the side member 820 of the shell 810(1) is coupled to the side member 820 of the shell 810(2).

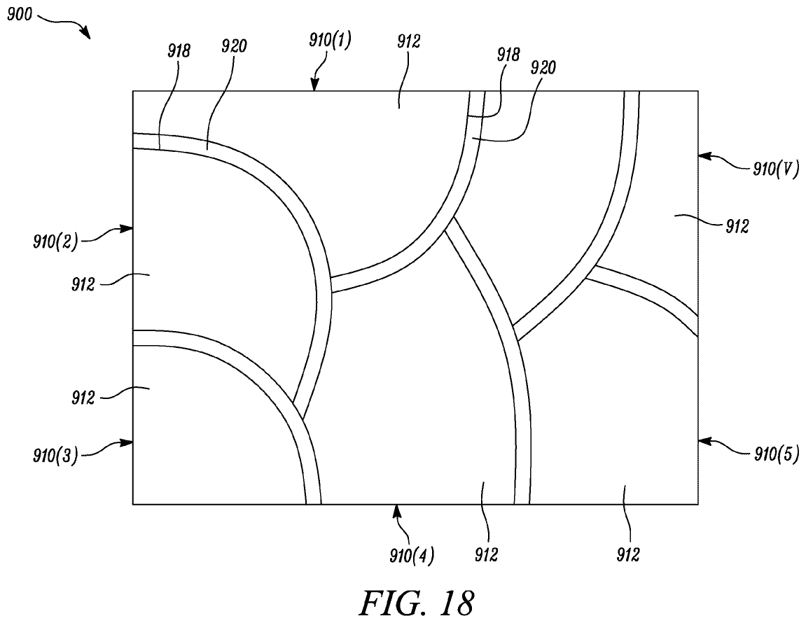

In some examples, the side member 820 of each of the plurality of shells 810(1)-810(U) may include one or more cutouts (e.g., the cutouts 250 shown in FIG. 4A) extending at least partially therethrough. The one or more cutouts may have any shape and size based on application requirements. FIG. 18 is a schematic top view of a portion of an article 900. In some examples, the article 900 may be functionally similar to the article 500 of FIGS. 10 and 11. The article 900 includes at least one shell 910 enclosing at least one adhesive layer (e.g., the adhesive layer 504 shown in FIG. 11). In the illustrated embodiment of FIG. 18, the at least one shell 910 includes a plurality of shells 910(1), 910(2), 910(V) (collectively, shells 910) connected to each other, where V is an integer corresponding to a total number of shells (e.g., V=10, 20, etc.). In the illustrated embodiment of FIG. 18, V=7.

In some examples, each of the plurality of shells 910(1)-910(V) includes a main member 912 and a perimeter 918. In the illustrated embodiment of FIG. 18, the main member 912 is irregularly shaped. However, it should be understood that the main member 912 may be of any shape based on application requirements. Each of the plurality of shells 910(1)-910(V) further includes a side member 920 extending from and coupled to at least a portion of the perimeter 918 of the main member 912. In some examples, each of the plurality of shells 910(1)-910(V) is at least partially coupled to at least one adjacent shell 910(1)-910(V) from the plurality of shells 910. For example, the shell 910(1) is coupled to the shell 910(2).

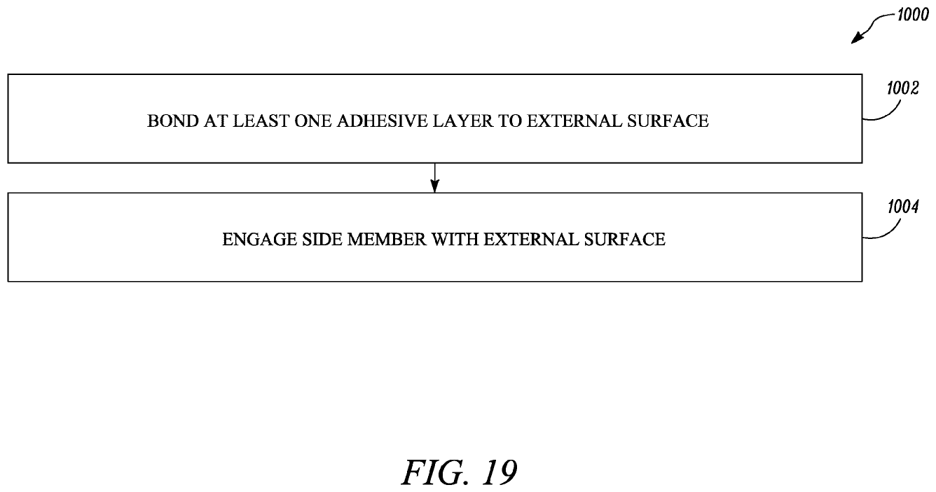

FIG. 19 is a flow chart illustrating a method 1000 of using the article 100, 200, 300, 400, 500, 600, 700. The method 1000 will be described with reference to the article 100, 200, 300, 400, 500, 600, 700 of FIGS. 1A-18.

Referring now to FIGS. 1A-19, at step 1002, the method 1000 includes bonding the at least one adhesive layer 104, 204, 304, 504, 604, 704 to the external surface 102, 402. At step 1004, the method 1000 further includes engaging the side member 120, 220, 320, 420, 520, 620, 720 with the external surface 102, 402.

In the article 100, 200, 300, 400, 500, 600, 700, 800, 900 of the present disclosure, at least one shell 110, 210, 310, 410, 510, 610, 710, 810, 910 may enclose the at least one adhesive layer 104, 204, 304, 504, 604, 704, 804 on the external surface 102, 402 to which the article 100, 200, 300, 400, 500, 600, 700, 800, 900 is attached through the main member 112, 212, 312, 412, 512, 612, 712, 812, 912 and the side member 120, 220, 320, 420, 520, 620, 720, 820, 920. Specifically, the side member 120, 220, 320, 420, 520, 620, 720, 820, 920 may at least partially surround the at least one adhesive layer 104, 204, 304, 504, 604, 704, 804 and may cover edges or a perimeter of the at least one adhesive layer 104, 204, 304, 504, 604, 704, 804. Thus, the at least one shell 110, 210, 310, 410, 510, 610, 710, 810, 910 may protect the at least one adhesive layer 104, 204, 304, 504, 604, 704, 804 from environmental factors, such as dirt, moisture, etc.

Moreover, the at least one shell 110, 210, 310, 410, 510, 610, 710, 810, 910 may also protect the edges or the perimeter of the at least one adhesive layer 104, 204, 304, 504, 604, 704, 804 from catching on external objects, such as clothing, thereby preventing the at least one adhesive layer 104, 204, 304, 504, 604, 704, 804 from being peeled up from the external surface 102, 402. Longer attachment period may improve a user experience and may enable less landfill waste.

The at least one shell 110, 210, 310, 410, 510, 610, 710, 810, 910 may also distribute lifting forces over a larger region, thereby mitigating the chances of edge lifting. Additionally, the side member 120, 220, 320, 420, 520, 620, 720, 820, 920 may flex relative to the main member 112, 212, 312, 412, 512, 612, 712, 812, 912. Thus, the at least one shell 110, 210, 310, 410, 510, 610, 710, 810, 910 may also protect the at least one adhesive layer 104, 204, 304, 504, 604, 704, 804 on the external surface 102, 402 that may be flexible and/or curved by allowing the side member 120, 220, 320, 420, 520, 620, 720, 820, 920 to at least partially surround the at least one adhesive layer 104, 204, 304, 504, 604, 704, 804 at all times.

In other words, having the flexible side member 120, 220, 320, 420, 520, 620, 720, 820, 920 extending from and coupled to at least the portion of the perimeter 118, 218, 318, 418, 518, 618, 718, 818, 918 of the main member 112, 212, 312, 412, 512, 612, 712, 812, 912 (which may be relatively rigid) may protect the at least one adhesive layer 104, 204, 304, 504, 604, 704, 804 even when the article 100, 200, 300, 400, 500, 600, 700, 800, 900 is attached to the external surface 102, 402 that may be flexible and/or curved. Thus, the article 100, 200, 300, 400, 500, 600, 700, 800, 900 of the present disclosure may provide a highly compliant attachment with longer attachment period and a better quality of life for the user.

The flexibility of the side member 120, 220, 320, 420, 520, 620, 720, 820, 920 relative to the main member 112, 212, 312, 412, 512, 612, 712, 812, 912 may be enhanced by varying a design of the side member 120, 220, 320, 420, 520, 620, 720, 820, 920, e.g., by making the side member 120, 220, 320, 420, 520, 620, 720, 820, 920 geometrically thinner, by adding tapers, or by adding notches.

Alternatively, or additionally, suitable materials may be chosen for the side member 120, 220, 320, 420, 520, 620, 720, 820, 920 that provide flexibility through intrinsic material properties.

In the present detailed description of the preferred embodiments, reference is made to the accompanying drawings, which illustrate specific embodiments in which the invention may be practiced. The illustrated embodiments are not intended to be exhaustive of all embodiments according to the invention. It is to be understood that other embodiments may be utilized, and structural or logical changes may be made without departing from the scope of the present invention. The following detailed description, therefore, is not to be taken in a limiting sense, and the scope of the present invention is defined by the appended claims.

Unless otherwise indicated, all numbers expressing feature sizes, amounts, and physical properties used in the specification and claims are to be understood as being modified in all instances by the term “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein.

As used in this specification and the appended claims, the singular forms “a,” “an,” and “the” encompass embodiments having plural referents, unless the content clearly dictates otherwise. As used in this specification and the appended claims, the term “or” is generally employed in its sense including “and/or” unless the content clearly dictates otherwise.

Spatially related terms, including but not limited to, “proximate,” “distal,” “lower,” “upper,” “beneath,” “below,” “above,” and “on top,” if used herein, are utilized for ease of description to describe spatial relationships of an element(s) to another. Such spatially related terms encompass different orientations of the device in use or operation in addition to the particular orientations depicted in the figures and described herein. For example, if an object depicted in the figures is turned over or flipped over, portions previously described as below or beneath other elements would then be above or on top of those other elements.

As used herein, when an element, component, or layer for example is described as forming a “coincident interface” with, or being “on,” “connected to,” “coupled with,” “stacked on” or “in contact with” another element, component, or layer, it can be directly on, directly connected to, directly coupled with, directly stacked on, in direct contact with, or intervening elements, components or layers may be on, connected, coupled or in contact with the particular element, component, or layer, for example. When an element, component, or layer for example is referred to as being “directly on,” “directly connected to,” “directly coupled with,” or “directly in contact with” another element, there are no intervening elements, components, or layers for example.

Various examples have been described. These and other examples are within the scope of the following claims.

1. An article, comprising:
at least one adhesive layer comprising an adhesive;
at least one tab connected to and extending from the at least one adhesive layer; and
at least one shell enclosing the at least one adhesive layer, the at least one shell comprising:
a main member comprising:
a first major surface;
a second major surface opposite to the first major surface, the second major surface coupled to the at least one adhesive layer; and
a perimeter disposed between the first major surface and the second major surface; and
a side member extending from and coupled to at least a portion of the perimeter of the main member, wherein the side member extends from the perimeter beyond the second major surface of the main member and at least partially surrounds the at least one adhesive layer, wherein the side member comprises one or more cutouts extending at least partially therethrough,
wherein the at least one tab extends through one of the one or more cutouts and is at least partially disposed outside of the at least one shell, and
wherein the side member is configured to flex relative to the main member.

2. (canceled)

3. (canceled)

4. The article of claim 1, wherein the perimeter comprises a first longitudinal edge extending along a longitudinal axis, a second longitudinal edge opposing the first longitudinal edge and extending along the longitudinal axis, a first transverse edge extending between the first longitudinal edge and the second longitudinal edge along a transverse axis orthogonal to the longitudinal axis, and a second transverse edge opposing the first transverse edge and extending between the first longitudinal edge and the second longitudinal edge along the transverse axis.

5. The article of claim 4, wherein the side member comprises a first side portion extending from the first longitudinal edge, a second side potion extending from the second longitudinal edge, a third side portion extending from the first transverse edge, and a fourth side portion extending from the second transverse edge, and wherein the first side portion, the second side portion, the third side portion, and the fourth side portion together at least partially surround the at least one adhesive layer.

6. The article of claim 5, wherein the one or more cutouts comprise:
a first cutout between the first side portion and the third side portion;
a second cutout between the second side portion and the third side portion;
a third cutout between the second side portion and the fourth side portion; and
a fourth cutout between fourth side portion and the first side portion, such that the first, second, third, and fourth side portions are spaced apart from each other.

7. The article of claim 1, wherein the side member tapers away from the perimeter.

8. The article of claim 1, wherein the side member comprises a side main portion extending from the perimeter and a curved lip extending from the side main portion opposite to the perimeter, and wherein the side main portion is hingedly coupled to the perimeter.

9. The article of claim 8, wherein the side main portion tapers away from the perimeter, such that the side main portion has a maximum thickness adjacent to the perimeter.

10. The article of claim 8, wherein the side main portion forms a living hinge.

11. The article of claim 1, wherein the side member comprises:
a side narrow portion disposed adjacent to the perimeter and hingedly coupled to the perimeter; and
a side main portion extending from the side narrow portion, wherein a thickness of the side narrow portion is less than a maximum thickness of the side main portion.

12. The article of claim 11, wherein the side narrow portion comprises at least one notch extending partially therethrough.

13. The article of claim 12, wherein the at least one notch comprises a first notch disposed proximal to the first major surface of the main member and a second notch aligned with and extending towards the first notch, and wherein the second notch is disposed distal to the first major surface of the main member.

14. The article of claim 11, wherein the side narrow portion forms a living hinge.

15. The article of claim 11, wherein the side main portion tapers away from the side narrow portion, such that the maximum thickness of the side main portion is adjacent to the side narrow portion.

16. The article of claim 11, wherein the side member further comprises a curved lip extending from the side main portion opposite to the perimeter.

17. The article of claim 1, further comprising a sensor disposed on the first major surface of the main member.

18. The article of claim 17, wherein the at least one shell further comprises a housing portion coupled to the main member and extending from the first major surface opposite to the at least one adhesive layer, and wherein the housing portion encloses the sensor.

19. The article of claim 17, wherein the main member further comprises a first aperture extending through the main member between the first major surface and the second major surface, wherein the at least one adhesive layer comprises a second aperture extending therethrough and at least partially aligned with the first aperture, and wherein the sensor further comprises a sensing portion extending at least partially through the first aperture and the second aperture.

20. The article of claim 1, wherein the main member has a shape selected from the group consisting of rectangular, square, circular, elliptical, oval, and polygonal.

21. The article of claim 1, wherein the main member is made from a first material and the side member is made from a second material different from the first material, and wherein an elastic modulus of the first material is greater than an elastic modulus of the second material by a factor of at least two.

22. The article of claim 1, wherein the main member and the side member are integrally formed.

23. The article of claim 1, wherein the adhesive is a pressure sensitive adhesive or a stretch-release adhesive.

24. The article of claim 1, wherein the at least one shell comprises a plurality of shells connected to each other, and wherein the side member of each of the plurality of shells is at least partially coupled to the side member of at least one adjacent shell from the plurality of shells.

25. The article of claim 24, wherein the at least one adhesive layer comprises a plurality of adhesive layers corresponding to the plurality of shells and spaced apart from each other, and wherein each adhesive layer from the plurality of adhesive layers is coupled to the second major surface of the main member of a corresponding shell from the plurality of shells.

26. The article of claim 24, wherein the plurality of shells are arranged in an array comprising a plurality of rows and a plurality of columns, and wherein the side member of each of the plurality of shells disposed in a corresponding row from the plurality of rows and a corresponding column from the plurality of columns is coupled to the side member of each adjacent shell disposed in the corresponding row and each adjacent shell disposed in the corresponding column.

27. The article of claim 1, wherein the at least one shell comprises a single shell, wherein the main member of the single shell comprises a plurality of main segments connected to each other, and wherein the plurality of main segments together define the perimeter.

28. The article of claim 27, the single shell further comprises a plurality of intermediate portions, wherein each of the plurality of intermediate portions connects a corresponding adjacent pair of main segments to each other, and wherein the plurality of main segments and the plurality of intermediate portions together define the first major surface and the second major surface.

29. The article of claim 28, wherein each of the plurality of intermediate portions comprises at least one notch extending partially therethrough.

30. The article of claim 28, wherein each of the plurality of main segments is made from a first material, wherein each of the plurality of intermediate portions is made from a third material different from the first material, and wherein an elastic modulus of the first material is greater than an elastic modulus of the third material by a factor of at least two.

31. The article of claim 28, wherein the at least one adhesive layer comprises a single adhesive layer disposed on the second major surface together defined by the plurality of main segments and the plurality of intermediate portions.

32. The article of claim 28, wherein at least the second major surface together defined by the plurality of main segments and the plurality of intermediate portions is planar.

33. The article of claim 27, wherein the at least one adhesive layer comprises a plurality of adhesive layers corresponding to the plurality of main segments and spaced apart from each other, and wherein each adhesive layer from the plurality of adhesive layers is coupled to a portion of the second major surface of a corresponding main segment from the plurality of main segments.

34. The article of claim 1, wherein the side member is hingedly coupled to at least the portion of the perimeter of the main member.

35. A method of using the article of claim 1, the method comprising:
bonding the at least one adhesive layer to an external surface; and
engaging the side member with the external surface.
